(19) United States
(12) Patent Application Publication
(10) Pub. No.: US 2017/0298318 A1
(43) Pub. Date: Oct. 19, 2017

(54) METHOD FOR PRODUCING A GRANULAR BIOMASS WHICH CONTAINS AN OXIDATION-SENSITIVE VALUABLE SUBSTANCE

(71) Applicant: Evonik Degussa GmbH, Essen (DE)

(72) Inventors: Christian Rabe, Grossostheim (DE); Mathias DERNEDDE, Bruchköbel (DE); Alexander SCHRICKEL, Gelnhausen (DE); Horst PRIEFERT, Ostbevern (DE); Stefan EILS, Gründau (DE); Michael DIEHL, Frankfurt (DE)

(73) Assignee: Evonik Degussa GmbH, Essen (DE)

(21) Appl. No.: 15/516,038

(22) PCT Filed: Sep. 22, 2015
(86) PCT No.: PCT/EP2015/071718

§ 371 Date: Mar. 31, 2017

(30) Foreign Application Priority Data
Oct. 2, 2014 (EP) 14187460.2

(51) Int. Cl. C12N 1/00; A61K 8/30; A23K 20/158; A61K 8/97; A61K 8/92; A61K 8/37; A61K 8/99; A23L 31/00

(52) U.S. Cl. CPC C12N 1/005; A61K 8/37; A61K 8/30; A23K 20/158; A61K 8/99; A61K 8/97; A61K 8/92; A23V 2002/00; A23L 31/00; C12N 1/00

(57) ABSTRACT

According to the invention, it has been found that a particulate biomass containing an oxidation-sensitive material of value can be converted into a particularly easy-to-handle product in a gentle manner if it is subjected to a granulation with the addition of an agglomeration auxiliary.

The present invention relates to a method for producing a granulated biomass which comprises an oxidation-sensitive material of value, and to the granulated biomass obtainable by this method.

The importance of microbial cells for producing materials of value is well known to those skilled in the art. An example of such materials of value are foodstuff components, in particular lipids, such as, for example, polyunsaturated fatty acids. A particular role is played in the production of such materials of value not only by bacteria and yeasts, but in particular also by other fungi and by algae.

Certain materials of value, in particular polyunsaturated fatty acids (PUFAs), are an important component for the nutrition of humans and animals. The source of PUFAs originally used was mostly fish. Furthermore, it is known that certain microbes are heterotrophic producers of PUFAs in large amounts, it being possible to influence, in an advantageous manner, the fatty acid production by selecting specific reaction parameters. The PUFAs can then be obtained from the cells, or else the cells can be used directly in feedstuffs or foodstuffs in the from of biomass.

In order to process the biomass for use in foodstuffs or feedstuffs, it is necessary to convert it into an easy-to-handle, flowable form.

It has been found, however, that the work-up of the biomass described in the prior art frequently leads to a difficult-to-handle, non-flowable, generally hygroscopic product. This is especially the case if the biomass comprises a high proportion of lipids, particularly triglycerides.

During spray drying of the biomass, a dusty and likewise difficulty pourable product is often formed.

It was therefore an object of the present invention to provide a method which allows the conversion of the particulate biomass to a easier-to-handle, flowable defined particulate product that is as dust-free as possible and also as non-hygroscopic as possible.

During the work-up, the material of value present should in particular also remain intact as far as possible. In this regard, the work-up should in particular also ensure that the cell membranes of the cells present in the biomass remain in intact form as far as possible in order to prevent the oxidative degradation of the material of value present. Furthermore, it should preferably also be prevented as far as possible that any liberated material of value is damaged, for example by oxidative degradation.

According to the invention, it has been found that the object according to the invention can be achieved by granulating a particulate starting biomass using an agglomeration auxiliary.

This procedure leads to a clearly defined, very easy-to-handle, flowable, dust-free and non-hygroscopic product which is highly suited for incorporation into foodstuffs or feedstuffs.

A first subject matter of the present invention is therefore a method for producing a particulate biomass which comprises an oxidation-sensitive material of value, characterized in that a particulate starting biomass is granulated using an agglomeration auxiliary or is subjected to a granulation method.

In accordance with the invention, “granulate” or “granulation” is understood to mean the transformation of a finely divided particulate powder into a coarse-grained particulate powder in which the coarse-grained powder obtainable preferably has a particle size of 0.1 to 2.0 mm (d50) and preferably has good flowability.

The present invention therefore also relates to a particulate biomass which can be obtained by a method according to the invention.

A further subject matter of the present invention is therefore also a particulate biomass which comprises an oxidation-sensitive material of value, characterized in that the particulate biomass comprises an agglomeration auxiliary.

The agglomeration auxiliary to be used according to the invention is preferably selected from optionally modified carbohydrates, proteins, further organic polymers, inorganic substances, and mixtures thereof.

Optionally modified carbohydrates that can be used are, for example, monomeric or oligomeric sugars, and mixtures thereof, in particular glucose, sucrose or maltodextrins. Preferably, the optionally modified carbohydrate is an optionally modified polysaccharide, the agglomeration auxiliaries used preferably being guaran, gum Arabic, guar gum, locust bean gum, xanthan gum, agar, carrageenan, starch, in particular cornstarch, tapioca starch or potato starch, cellulose or its derivatives, hemicellulose or its derivatives, alginic acid or maltodextrin, and mixtures thereof.

In accordance with the invention, “derivatives” of carbohydrates and “modified” carbohydrates are understood to mean in particular carbohydrates modified by alkyl, particularly C₁₋₆-alkyl, especially C₁₋₄-alkyl groups, by carboxyalkyl, particularly carboxy-C₁₋₆-alkyl, especially carboxy-C₁₋₄-alkyl groups, and also by hydroxyalkyl, particularly hydroxy-C₁₋₆-alkyl, especially hydroxy-C₁₋₄-alkyl groups.

As agglomeration auxiliary, particular preference is given to using modified cellulose, in particular cellulose modified by carboxy groups, in particular carboxymethylcellulose and/or hydroxypropylmethylcellulose, very particularly preferably carboxymethylcellulose. The preferably used sodium salt of carboxymethylcellulose is obtainable for example by reaction of sodium chloroacetate with alkalicellulose and is commercially available under the trade name Blanose (Ashland, USA).

Modified celluloses that can be used according to the invention, in particular carboxymethylcelluloses, preferably have an average molecular weight of from 80000 to 80000 g/mol, in particular 90000 to 700000 g/mol, particularly preferably 100000 to 600000 g/mol, in particular 150000 to 400000 g/mol, in particular 200000 to 300000 g/mol.

Maltodextrins are likewise particularly preferably used as agglomeration auxiliary. Maltodextrins are water-soluble carbohydrate mixtures which are obtainable by hydrolysis of starch. They are a mixture of monomers, dimers, oligomers and polymers of glucose. The percentage composition differs depending on the degree of hydrolysis. The degree of hydrolysis is given in dextrose equivalents (DE). Starch has a DE value of 1, glucose has a DE value of 100. Maltodextrins have a DE value of 3 to 20. According to the invention, maltodextrins with any desired DE values can be used, but preference is given to using a maltodextrin with a DE value of 3 to 10.

Furthermore, proteins can be used as agglomeration auxiliaries, in particular casein, gelatin, collagen, wheat gluten, and mixtures thereof.

Furthermore, organic polymers can be used as agglomeration auxiliary, in particular lignosulphonates, polymethylolcarbamides, polyacrylic acids or polyvinyl alcohols, and mixtures thereof.

Furthermore, inorganic substances can be used as agglomeration auxiliary, in particular silicates, preferably bentonites, hexametaphosphate, and mixtures thereof. The silicate here is preferably a neutral silicate.

The granulation is preferably carried out according to the invention at a product temperature of 40-60° C. The granulation can be carried out using standard commercial granulators, for example using moving-bed granulators, in particular using spouted-bed, sieve-plate or fluidized-bed granulators. Granulators that can be used according to the invention are sold, for example by Glatt (Binzen, Germany). By way of example, mention may be made of the granulator ProCell-LabSystem (Glatt, Germany).

The agglomeration of the spray-dried particles preferably takes place in a fluidized-bed method using the aforementioned granulators. For this, the fine, preferably spray-dried, powder is introduced into the fluidized bed, and the agglomeration auxiliary—preferably in dissolved form, for example as an aqueous solution—is sprayed in and therefore finely distributed in the fluidized bed. The solvent is preferably evaporated by heating the incoming air. The incoming air rate here is preferably adjusted such that a fluidization of the agglomerated particles is possible. This is achieved by regulating the amount of incoming air. When the agglomerates achieve the desired particle size, they are no longer fluidized on account of their weight and can be removed (classified out) at the lower end of the fluidized bed and/or at the side outlet of the continuous fluidized bed. The residence time of the particles in the fluidized bed is thus adjusted via the rate of particle growth since the particles have to grow from the fine, spray-dried particle to the relatively large agglomerate in order to leave the fluidized bed. The conditions to be applied correspond to the standard conditions of a thermal granulation.

In a biomass granulate according to the invention, the agglomeration auxiliary is present preferably in an amount of from 0.05 to 6% by weight, in particular 0.1 to 5.0% by weight, preferably 0.2 to 4.5% by weight, in particular 0.3 to 4.0% by weight, particularly preferably 0.4 to 3.5% by weight, in particular 0.5 to 3.2% by weight, especially 0.8 to 3.0% by weight, in particular 1.0 to 2.5% by weight.

A biomass granulate particularly preferred according to the invention comprises carboxymethylcellulose in an amount of from 0.05 to 6% by weight, in particular 0.1 to 5% by weight, preferably 0.2 to 4.5% by weight, in particular 0.3 to 4.0% by weight, particularly preferably 0.4 to 3.5% by weight, in particular 0.5 to 3.2% by weight, especially 0.8 to 3.0% by weight, in particular 1.0 to 2.5% by weight.

A further biomass granulate particularly preferred according to the invention comprises maltodextrins in an amount of from 0.05 to 6% by weight, in particular 0.1 to 5% by weight, preferably 0.2 to 4.5% by weight, in particular 0.3 to 4.0% by weight, particularly preferably 0.4 to 3.5% by weight, in particular 0.5 to 3.2% by weight, especially 0.8 to 3.0% by weight, in particular 1.0 to 2.5% by weight.

The agglomeration auxiliary is in each case accordingly metered in methods according to the invention in an amount such that the stated % by weight fraction of agglomeration auxiliary in the formed granulate is established.

The biomass granulate obtained upon carrying out the granulation is preferably characterized in that the average particle diameter (d50) is 150 to 1000 μm.

The biomass granulate obtained upon carrying out the granulation is further preferably characterized in that at least 80% by weight, in particular at least 90% by weight, particularly preferably at least 95% by weight, especially at least 98% by weight, of the particles have a particle diameter (grain size) of from 100 to 2000 μm, preferably 100 to 1500 μm, particularly preferably 100 to 1000 μm.

The particulate biomass which is used in the granulation method according to the invention can be obtained for example starting from fermentation broth containing biomass by freeze drying or drum drying.

However, it has proven to be particularly advantageous according to the invention if particulate biomass which has been obtained by spray drying is used in the granulation method according to the invention. In this connection, preference is given to using fermentation broth, optionally after prior concentration, which accordingly contains biomass comprising an oxidation-sensitive material of value in the spray drying. The spray drying can take place in a manner known to the person skilled in the art, for example using a spray tower. In particular, a jet-spray drying, in particular using a single-substance nozzle or two-substance nozzle, or drying using a rotating disc can be carried out. By way of example, mention may be made of the spray dryer Production Minor™ Spray Dryer (GEA Niro, Müllheim, Germany).

In a preferred embodiment, during the spray drying of the biomass for the purposes of obtaining the particulate starting biomass, a hydrophilic or hydrophobic silicate is used. Alternatively or additionally, the hydrophilic or hydrophobic silicate can also be admixed after the spray drying or also after the agglomeration.

The silicate, if used, is preferably used in an amount such that in the final granulation product a concentration of from 0.05 to 5% by weight, in particular 0.1 to 4% by weight, preferably 0.15 to 3.5% by weight, in particular 0.2 to 3.0% by weight, particularly preferably 0.25 to 2.5% by weight, in particular 0.3 to 2.0% by weight, especially 0.35 to 1.8% by weight, in particular 0.4 to 1.5% by weight, is established.

In a further embodiment preferred according to the invention, a biomass granulate according to the invention is therefore characterized in that, besides the agglomeration auxiliary, it also comprises silicate, in particular hydrophobic and/or hydrophilic silicate, preferably in the amount stated above.

The particulate starting biomass is produced according to the invention preferably starting from a fermentation broth which contains the biomass.

Before the spray drying of the fermentation broth containing the biomass, a concentration of the fermentation broth containing the biomass can also initially take place in order to increase the solids content prior to the spray drying. However, prior concentration is not strictly necessary.

If hydrophilic or hydrophobic silicate is used during the spray drying, the silicate is preferably only mixed during the drying step or in the course of the drying method with the fermentation broth or the still-wet biomass. In the latter case, the silicate is preferably metered into the drying zone using a nozzle, preferably a two-material nozzle. In the case of nozzle spray drying, the drying zone is the zone beneath the spraying-in nozzle through which the fermentation broth is metered in. The silicate here can be added in suspended form, but is preferably metered in dry, in particular pulverulent, form.

Hydrophilic silicas are registered under CAS No. 112926-00-8 and are commercially available for example under the trade name Sipernat® (Evonik Industries, Germany).

Hydrophilic silicas preferably used according to the invention have a specific surface area (ISO 9277) of 130 to 600 m²/g, preferably 160 to 550 m²/g, and preferably have a dioctyl adipate absorption value of 1.5-4.0 ml/g, preferably 2.0-3.2 ml/g. They preferably also have a tamped density (unsieved; based on ISO 787-11) of 80 to 300 g/l, preferably 100 to 270 g/l. The particle size of the hydrophilic silica (d50; laser diffraction; based on ISO 13320-1) is preferably 10 to 150 μm, particularly 15 to 130 μm. Loss on drying of the hydrophilic silica (2 hours at 105° C.; based on ISO 787-2) is preferably at most 10%, particularly preferably at most 7%. Ignition loss of the hydrophobic silica (2 hours at 1000° C.; based on ISO 3262-1) is preferably at most 10%, particularly preferably at most 6%. The silicon dioxide content of the hydrophilic silica is preferably at least 95% by weight, particularly preferably at least 97% by weight (based on ISO 3262-19). The pH of the hydrophilic silica (5% in water; based on ISO 787-9) is preferably from 5.0 to 7.0, particularly preferably from 6.0 to 6.5.

In a particularly preferred embodiment according to the invention, the hydrophilic silica is a product having a specific surface area of 160 to 220 m²/g, a dioctyl adipate absorption value of 2.0-2.8 ml/g, a tamped density of 200 to 300 g/l and a particle size of 100 to 150 μm. Such a product is obtainable commercially under the name of Sipernat® 22 S (Evonik Industries, Germany).

In a further particularly preferred embodiment according to the invention, the hydrophilic silica is a product having a specific surface area of 450 to 550 m²/g, a dioctyl adipate absorption value of 2.5-3.5 ml/g, a tamped density of 80 to 130 g/l and a particle size of 10 to 40 μm. Such a product is obtainable commercially under the name of Sipernat® 50 S (Evonik Industries, Germany).

Hydrophobic silicas are registered under CAS No. 68611-44-9 and are also commercially available, for example, under the trade name Sipernat® (Evonik Industries, Germany). Hydrophobic silicas preferably used according to the invention have a methanol wettability of at least 40%, preferably at least 45%, particularly preferably at least 50%, particularly 40 to 65%, especially 50 to 60%.

They preferably also have a tamped density (unsieved; based on ISO 787-11) of 100 to 200 g/l, preferably 125 to 175 g/l. The particle size of the hydrophobic silica (d50; laser diffraction; based on ISO 13320-1) is preferably 5 to 15 μm, particularly 8 to 12 μm. Loss on drying of the hydrophobic silica (2 hours at 105° C.; based on ISO 787-2) is preferably at most 10%, particularly preferably at most 6%. Ignition loss of the hydrophobic silica (2 hours at 1000° C.; based on ISO 3262-1) is preferably at most 10%, particularly preferably at most 6%. The silicon dioxide content of the hydrophobic silica is preferably at least 95% by weight, particularly preferably at least 97% by weight (based on ISO 3262-19). The carbon content of the hydrophobic silica is preferably at most 3.5% by weight, particularly at most 2% by weight (based on ISO 3262-19). The pH of the hydrophobic silica (5% in a 1:1 mixture of water and methanol; based on ISO 787-9) is preferably from 7 to 10.5, particularly preferably from 7.5 to 9.

The methanol wettability is a measure of the hydrophobicity of the silica powder. To determine this value, a certain amount of silica powder is weighed into water. The silica powder remains here on the surface. The amount of methanol required for wetting the powder is then determined. “Methanol wettability” is here understood to mean the methanol content of a methanol-water mixture in % by volume in which 50% of the hydrophobic silica sediments.

Hydrophobic silicas which can be used in accordance with the invention are, for example, obtainable under the trade names Sipernat® D 10, Sipernat® D 15 and Sipernat® D 17 (Evonik Industries, Germany).

Prior to start of the spray drying, the fermentation broth used during the spray drying preferably has a solids content of from 10 to 50% by weight, and accordingly a water content of from 50 to 90% by weight. If required, the fermentation broth is adjusted to this water content prior to the actual drying. This may be carried out in particular by centrifugation, flotation, filtration, particularly ultrafiltration or microfiltration, decanting and/or solvent evaporation. In this case the solvent is preferably evaporated using a rotary evaporator, a thin film evaporator or a falling-film evaporator in a single stage or multistage process. Alternatively, reverse osmosis, for example, is also useful for concentrating the fermentation broth.

In this first optional but preferred step, the fermentation broth is preferably concentrated to a solids content of at least 10 or 15% by weight, preferably of at least 20 or 25% by weight, particularly 10 to 50 or 15 to 45% by weight, particularly preferably 15 to 40% by weight or 20 to 40% by weight.

This means the biomass to be dried to give the particulate starting biomass is preferably present prior to the spray drying in the form of a suspension having the solids fraction stated above, the suspension preferably being a fermentation broth or concentrated fermentation broth.

After the optional concentration of the fermentation broth, the drying of the biomass now preferably takes place by spray drying, in particular by nozzle spray drying or spray drying using a rotating disc.

Optionally, the biomass may also be subjected to the drying step directly after harvesting without prior concentration, particularly if the fermentation broth obtained already has a high solids content, preferably as stated above.

As a result of the primary drying, preferably spray drying, of the biomass, this is preferably dried to a residual moisture content of at most 10% by weight, particularly 0 to 10% by weight, particularly preferably at most 8% by weight, particularly 0.5 to 8% by weight, above all at most 6 or 5% by weight, particularly 0.5 to 6 or 0.5 to 5% by weight.

In the nozzle spray drying process, the fermentation broth introduced is atomized in a defined droplet size and is dried with the drying air introduced in a continuous flow. Since the individual drops are separate from one another in this method, there is good heat and mass transfer and thus an efficient drying. Moreover, the particle size of the dried end product can be adjusted in a defined manner via the established droplet size in the nozzle.

If hydrophilic or hydrophobic silicate is used in the spray drying, then this is preferably atomized in the drying zone in order to avoid the drying particles sticking together. The silicate thus functions as a so-called anticaking agent, thus facilitating the setting of a defined and controllable particle size.

In order to largely avoid the oxidation of oxidation-sensitive material of value, the drying gas during the spray drying can if desired be passed over the biomass in cycle gas mode. “Cycle gas mode” means that the gas used for the drying is passed over the biomass in a circulating manner. The gas used in the drying process preferably has a temperature above the saturation temperature of the solvent to be evaporated. The gas used is preferably air, particularly preferably air with a reduced content of oxygen.

The gas conducted in cycle gas mode preferably has an oxygen content of less than 20% by weight, preferably less than 15% by weight, particularly from 5 to 13% by weight.

The gas is preferably generated by passing air over a burner and heating it in this manner. The oxygen content of the air is thereby reduced at the same time to less than 20% by weight, preferably to less than 15% by weight, particularly from 5 to 13% by weight. The gas is constantly readjusted in the same manner in order to generate a constant gas flow with reduced oxygen content.

The drying temperature in the spray nozzle tower can be set to 95° C. owing to the short residence times.

“Solids content” in accordance with the invention is understood to mean the mass which remains on complete removal of the water. This dry mass also includes, in addition to suspended substances if applicable (such as the biomass), dissolved substances which only crystallize out or precipitate on drying. The solids content is in this respect complementary to the water or moisture content.

The composition comprising biomass used in the drying process is preferably the product of a cultivation process by fermentation and is also correspondingly referred to as fermentation broth. The fermentation broth to be used according to the invention preferably comprises further constituents of the fermentation medium in addition to the biomass to be dried. These constituents may take the form of, in particular, salts, antifoam agents and unreacted carbon source and/or nitrogen source. In the drying process, a product is preferably formed having a cell content of at least 60% by weight, preferably at least 65% by weight, particularly at least 70 or 80% by weight, where the further constituents present are the silica and optionally the further constituents of the fermentation medium mentioned above and also optionally components liberated partially from the cells. The further constituents of the fermentation broth may optionally be partially removed prior to drying the biomass, for example, by solid-liquid separation methods, such that a product is formed in the drying process that preferably comprises these further components of the fermentation broth, particularly salts, preferably in an amount of at most 20% by weight, particularly at most 15, 10 or 5% by weight.

The cells present in the biomass are preferably cells comprising a material of value, preferably an oxidation-sensitive material of value. These can particularly take the form of cells which already naturally produce materials of value, preferably lipids, in particular PUFAs (polyunsaturated fatty acids), but may also take the form of cells which have been made capable of producing lipids, in particular PUFAs, by means of suitable genetic engineering methods. In this context, the production may be autotrophic, mixotrophic or heterotrophic.

The biomass preferably comprises cells which produce lipids, in particular PUFAs, heterotrophically. The cells according to the invention preferably take the form of algae, fungi, particularly yeasts, or protists. The cells are especially preferably microbial algae or fungi.

Suitable cells of oil-producing yeasts are, in particular, strains of Yarrowia, Candida, Rhodotorula, Rhodosporidium, Cryptococcus, Trichosporon and Lipomyces.

The biomass according to the invention preferably comprises cells from the taxon Labyrinthulomycetes (Labyrinthulea, slime nets), in particular those of the family of Thraustochytriaceae. The family of the Thraustochytriaceae includes the genera Althomia, Aplanochytrium, Elnia, Japonochytrium, Schizochytrium, Thraustochytrium, Aurantiochytrium, Oblongichytrium and Ulkenia. The biomass particularly preferably comprises cells from the genera Thraustochytrium, Schizochytrium, Aurantiochytrium or Oblongichytrium, particularly those from the genus Aurantiochytrium.

Within the genus Aurantiochytrium, according to the invention the species Aurantiochytrium limacinum (previously also called Schizochytrium limacinum) is preferred. According to the invention, the strain Aurantiochytrium limacinum SR21 (IFO 32693) is used with very particular preference.

The oxidation-sensitive material of value is preferably an oxidation-sensitive lipid, particularly an unsaturated fatty acid, particularly preferably a polyunsaturated fatty acid (PUFA) or highly-unsaturated fatty acid (HUFA).

The cells present in the biomass are preferably distinguished by the fact that they contain at least 20% by weight, preferably at least 30% by weight, in particular at least 40% by weight, of material of value, preferably of lipids, especially preferably of PUFAs, in each case based on cell dry matter.

In a preferred embodiment, the majority of the lipids in this case is present in the form of triglycerides, with preferably at least 50% by weight, in particular at least 75% by weight and, in an especially preferred embodiment, at least 90% by weight of the lipids present in the cell being present in the form of triglycerides.

Furthermore, the lipids present in the cell preferably comprise polyunsaturated fatty acids (PUFAs), with preferably at least 10% by weight, in particular at least 20% by weight, especially preferably 20 to 60% by weight, in particular 20 to 40% by weight, of the fatty acids present in the cell being PUFAs.

According to the invention, polyunsaturated fatty acids (PUFAs) are understood to mean fatty acids having at least two, particularly at least three, C—C double bonds. According to the invention, highly-unsaturated fatty acids (HUFAs) are preferred among the PUFAs. According to the invention, HUFAs are understood to mean fatty acids having at least four C—C double bonds.

The PUFAs may be present in the cell in free form or in bound form. Examples of the presence in bound form are phospholipids and esters of the PUFAs, in particular monoacyl-diacyl- and triacylglycerides. In a preferred embodiment, the majority of the PUFAs is present in the form of triglycerides, with preferably at least 50% by weight, in particular at least 75% by weight and, in an especially preferred embodiment, at least 90% by weight of the PUFAs present in the cell being present in the form of triglycerides.

Preferred PUFAs are omega-3 fatty acids and omega-6 fatty acids, with omega-3 fatty acids being especially preferred. Preferred omega-3 fatty acids here are the eicosapentaenoic acid (EPA, 20:5ω-3), particularly the (5Z,8Z,11Z,14Z,17Z)-eicosa-5,8,11,14,17-pentaenoic acid, and the docosahexaenoic acid (DHA, 22:6ω-3), particularly the (4Z,7Z,10Z,13Z,16Z,19Z)-docosa-4,7,10,13,16,19-hexaenoic acid, with the docosahexaenoic acid being especially preferred.

Methods for producing the biomass, in particular that biomass which comprises cells containing lipids, in particular PUFAs, particularly of the order Thraustochytriales, are described in detail in the prior art (see e.g. WO91/07498, WO94/08467, WO97/37032, WO97/36996, WO01/54510). As a rule, the production takes place by cells being cultured in a fermenter in the presence of a carbon source and of a nitrogen source. In this context, biomass densities of more than 100 grams per litre and production rates of more than 0.5 gram of lipid per litre per hour may be attained. The process is preferably carried out in what is known as a fed-batch process, i.e. the carbon and nitrogen sources are fed in incrementally during the fermentation. When the desired biomass has been obtained, lipid production may be induced by various measures, for example by limiting the nitrogen source, the carbon source or the oxygen content or combinations of these.

Preferably, the cells are fermented in a medium with low salinity, in particular so as to avoid corrosion. This can be achieved by using chlorine-free sodium salts as the sodium source instead of sodium chloride, such as, for example, sodium sulphate, sodium carbonate, sodium hydrogen carbonate or soda ash. Preferably, chloride is used in the fermentation in amounts of less than 3 g/l, in particular less than 500 mg/l, especially preferably less than 100 mg/l.

Suitable carbon sources are both alcoholic and non-alcoholic carbon sources. Examples of alcoholic carbon sources are methanol, ethanol and isopropanol. Examples of non-alcoholic carbon sources are fructose, glucose, sucrose, molasses, starch and corn syrup.

Suitable nitrogen sources are both inorganic and organic nitrogen sources. Examples of inorganic nitrogen sources are nitrates and ammonium salts, in particular ammonium sulphate and ammonium hydroxide. Examples of organic nitrogen sources are amino acids, in particular glutamate, and urea.

In addition, inorganic or organic phosphorus compounds and/or known growth-stimulating substances such as, for example, yeast extract or corn steep liquor, may also be added so as to have a positive effect on the fermentation.

The cells are preferably fermented at a pH of 3 to 11, in particular 4 to 10, and preferably at a temperature of at least 20° C., in particular 20 to 40° C., especially preferably at least 30° C. A typical fermentation process takes up to approximately 100 hours.

After the fermentation has ended, the biomass is harvested. After harvesting the biomass or even optionally shortly before harvesting the biomass, the cells are preferably pasteurized in order to kill the cells and to deactivate enzymes which might promote lipid degradation. The pasteurization is preferably effected by heating the biomass to a temperature of 50 to 121° C. for a period of 5 to 60 minutes.

Likewise, after harvesting the biomass or even optionally shortly before harvesting the biomass, antioxidants are preferably added in order to protect the material of value present in the biomass from oxidative degradation. Preferred antioxidants in this context are BHT, BHA, TBHA, ethoxyquin, beta-carotene, vitamin E and vitamin C. The antioxidant, if used, is preferably added in an amount of 0.01 to 2% by weight.

According to the invention, particular preference is given to a method for producing a particulate biomass, characterized in that a particulate starting biomass is granulated in the presence of an agglomeration auxiliary, such that the agglomeration auxiliary is present in the final product in a concentration of from 0.1 to 6% by weight, preferably 0.2 to 5% by weight, particularly preferably 0.5 to 4% by weight, especially 0.8 to 3% by weight, and where the biomass contains cells from the taxon Labyrinthulomycetes, in particular those of the family of Thraustochytriaceae, particularly those of the genera Thraustochytrium, Schizochytrium, Aurantiochytrium or Oblongichytrium.

Very particular preference is given here to a method for producing a particulate biomass, characterized in that a particulate starting biomass is granulated in the presence of an optionally modified polysaccharide as agglomeration auxiliary, such that the optionally modified polysaccharide is present in the final product in a concentration of from 0.1 to 6% by weight, preferably 0.2 to 5% by weight, particularly preferably 0.5 to 4% by weight, especially 0.8 to 3% by weight, and where the biomass contains cells from the taxon Labyrinthulomycetes, in particular those of the family of Thraustochytriaceae, especially those of the genera Thraustochytrium, Schizochytrium, Aurantiochytrium or Oblongichytrium.

To a quite particular degree, preference is given here to a method for using a particulate biomass, characterized in that a particulate starting biomass is granulated in the presence of carboxymethylcellulose as agglomeration auxiliary, such that the carboxymethylcellulose is present in the final product in a concentration of from 0.1 to 6% by weight, preferably 0.2 to 5% by weight, particularly preferably 0.5 to 4% by weight, especially 0.8 to 3% by weight, and where the biomass contain cells from the taxon Labyrinthulomycetes, in particular those of the family of Thraustochytriaceae, especially those of the genera Thraustochytrium, Schizochytrium, Aurantiochytrium or Oblongichytrium, in particular the species Aurantiochytrium limacinum, especially the strain Aurantiochytrium limacinum SR21.

To a very particular extent, preference is likewise given to a method for producing a particulate biomass, characterized in that a particulate starting biomass is granulated in the presence of a maltodextrin, in particular a maltodextrin with a DE value of 3 to 10, as agglomeration auxiliary, such that the maltodextrin is present in the final product in a concentration of from 0.1 to 6% by weight, preferably 0.2 to 5% by weight, particularly preferably 0.5 to 4% by weight, especially 0.8 to 3% by weight, and where the biomass contains cells from the taxon Labyrinthulomycetes, in particular those of the family of Thraustochytriaceae, especially those of the genera Thraustochytrium, Schizochytrium, Aurantiochytrium or Oblongichytrium, in particular the species Aurantiochytrium limacinum, especially the strain Aurantiochytrium limacinum SR21.

The particles produced by granulation methods according to the invention have excellent strength and, on account of their low caking tendency, have very good bulk properties and flow characteristics. Moreover, the particles have a low residual moisture and are preferably low-dust.

A free-flowing particulate product is thus obtained by the granulation. A product having the desired particle size can optionally be obtained from the granulate obtained by sieving or dust separation.

“Free-flowing” according to the invention is understood to mean a powder that can flow out unhindered from a series of glass efflux vessels having different size outflow openings, at least from the vessel having the 5 millimetre opening (Klein: Seifen, Öle, Fette, Wachse 94, 12 (1968)).

“Fine-grained” according to the invention is understood to mean a powder having a predominant fraction (>50%) of particle sizes of 20 to 100 micrometres in diameter.

“Coarse-grained” according to the invention is understood to mean a powder having a predominant fraction (>50%) of particle sizes of 100 to 2500 micrometres in diameter.

“Dust-free” according to the invention is understood to mean a powder which contains only low fractions (<10% by weight, preferably <5% by weight, in particular <3% by weight, especially <1% by weight) of particle sizes below 100 micrometres.

Grain or particle size is preferably determined according to the invention by laser diffraction spectrometric methods. Possible methods are described in the text book “Teilchengröβenmessung in der Laborpraxis” [Particle size measurement in the laboratory] by R. H. Müller and R. Schuhmann, Wissenschaftliche Verlagsgesellschaft Stuttgart (1996) and in the text book “Introduction to Particle Technology” by M. Rhodes, Wiley & Sons (1998). Inasmuch as various methods can be used, the first-cited usable method from the text book of R. H. Müller and R. Schuhmann for the measuring of particle size is preferably used.

The products obtained by granulation methods according to the invention preferably have a fraction of at least 80% by weight, in particular at least 90% by weight, particularly preferably at least 95% by weight, especially at least 98% by weight of particles with a particle size of from 100 to 2000 micrometres, preferably 100 to 1500 micrometres, in particular 100 to 1000 micrometres.

The particulate starting biomass preferably obtainable by spray drying methods, by contrast, preferably has a fraction of at least 80% by weight, in particular at least 90% by weight, particularly preferably at least 95% by weight, of particles with a particle size of 100 to 500 micrometres, preferably 100 to 400 micrometres, especially 100 to 300 micrometres.

On account of the production method, a structure is formed which is recognisable as an agglomerate of smaller particles. Preferably, at least 50 or 60% by weight, in particular at least 70 or 80% by weight, particularly preferably at least 90 or 95% by weight, especially essentially all of the particles have such an agglomerate structure.

At the same time, the particles according to the invention have a very low caking tendency. This is presumably attributed to the reduction, associated with the agglomeration, in the surface-to-volume ratio of the particles. In this respect, in a preferred embodiment, it is also possible to dispense entirely with the explicit addition of anticaking agents—such as the aforementioned silicate—for the purpose of retaining a free-flowing product.

The fraction of dust, i.e. particles with a particle size of less than 100 micrometres, is preferably at most 10% by weight, in particular at most 8 or 6% by weight, particularly preferably at most 4% by weight, in particular at most 2% by weight.

The bulk density of the products according to the invention is preferably 350 to 550 kg/m³, particularly preferably 350 to 500 kg/m³, in particular 350 to 450 kg/m³.

The particles with an agglomerate structure preferably have a non-spherical geometry. In a preferred embodiment, therefore, at least 50 or 60% by weight, in particular at least 70 or 80% by weight, particularly preferably at least 90 or 95% by weight, primarily essentially all particles, are present in non-spherical form.

“Non-spherical” is preferably understood here to mean that the diameter of a particle starting from the mass centre of the particle is not the same in all spatial directions. Particularly preferably, the deviation of the diameter of a particle starting from the mass centre of the particle is at least with regard to two spatial directions at least 20%, preferably at least 25%, particularly preferably at least 30%.

A particularly preferred subject matter of the present invention is a particulate biomass which comprises an agglomeration auxiliary in an amount of from 0.05 to 6% by weight, in particular 0.1 to 5% by weight, preferably 0.2 to 4.5% by weight, in particular 0.4 to 4.0% by weight, particularly preferably 0.6 to 3.5% by weight, in particular 0.8 to 3.2% by weight, especially 1.0 to 3.0% by weight, in particular 1.5 to 2.5% by weight, and also cells from the taxon Labyrinthulomycetes, in particular those of the family of Thraustochytriaceae, especially those of the genera Thraustochytrium, Schizochytrium, Aurantiochytrium or Oblongichytrium, in particular the species Aurantiochytrium limacinum, especially the strain Aurantiochytrium limacinum SR21, where at least 80% by weight, preferably at least 90% by weight, preferably at least 95% by weight, of the particles present have a particle size of from 100 to 2000 micrometres, preferably 100 to 1500 micrometres, especially 100 to 1000 micrometres.

Preference is given here in particular to a particulate biomass which comprises an optionally modified polysaccharide in an amount of from 0.05 to 6% by weight, in particular 0.1 to 5% by weight, preferably 0.2 to 4.5% by weight, in particular 0.4 to 4.0% by weight, particularly preferably 0.6 to 3.5% by weight, in particular 0.8 to 3.2% by weight, especially 1.0 to 3.0% by weight, in particular 1.5 to 2.5% by weight, and also cells from the taxon Labyrinthulomycetes, in particular those of the family of Thraustochytriaceae, especially those of the genera Thraustochytrium, Schizochytrium, Aurantiochytrium or Oblongichytrium, in particular the species Aurantiochytrium limacinum, especially the strain Aurantiochytrium limacinum SR21, where at least 80% by weight, in particular at least 90% by weight, preferably at least 95% by weight, of the particles present have a particle size of from 100 to 2000 micrometres, preferably 100 to 1500 micrometres, especially 100 to 1000 micrometres.

Preference is furthermore given in particular in this connection to a particulate biomass which comprises modified cellulose, in particular carboxymethylcellulose, in an amount of from 0.05 to 6% by weight, in particular 0.1 to 5% by weight, preferably 0.2 to 4.5% by weight, in particular 0.4 to 4.0% by weight, particularly preferably 0.6 to 3.5% by weight, in particular 0.8 to 3.2% by weight, especially 1.0 to 3.0% by weight, in particular 1.5 to 2.5% by weight, and also cells from the taxon Labyrinthulomycetes, in particular those of the family of Thraustochytriaceae, especially those of the genera Thraustochytrium, Schizochytrium, Aurantiochytrium or Oblongichytrium, in particular the species Aurantiochytrium limacinum, especially the strain Aurantiochytrium limacinum SR21, where at least 80% by weight, in particular at least 90% by weight, preferably at least 95% by weight, of the particles present have a particle size of from 100 to 2000 micrometres, preferably 100 to 1500 micrometres, especially 100 to 1000 micrometres.

Preference is given in particular also to a particulate biomass which comprises a maltodextrin, in particular a maltodextrin with a DE value of 3 to 10, in an amount of from 0.05 to 6% by weight, in particular 0.1 to 5% by weight, preferably 0.2 to 4.5% by weight, in particular 0.4 to 4.0% by weight, particularly preferably 0.6 to 3.5% by weight, in particular 0.8 to 3.2% by weight, especially 1.0 to 3.0% by weight, in particular 1.5 to 2.5% by weight, and also cells from the taxon Labyrinthulomycetes, in particular those of the family of Thraustochytriaceae, especially those of the genera Thraustochytrium, Schizochytrium, Aurantiochytrium or Oblongichytrium, in particular the species Aurantiochytrium limacinum, especially the strain Aurantiochytrium limacinum SR21, where at least 80% by weight, in particular at least 90% by weight, preferably at least 95% by weight, of the particles present have a particle size of from 100 to 2000 micrometres, preferably 100 to 1500 micrometres, especially 100 to 1000 micrometres.

A particular subject matter of the present invention is a particulate biomass which comprises an optionally modified polysaccharide in an amount of from 0.05 to 6% by weight, in particular 0.1 to 5% by weight, preferably 0.2 to 4.5% by weight, in particular 0.4 to 4.0% by weight, particularly preferably 0.6 to 3.5% by weight, in particular 0.8 to 3.2% by weight, especially 1.0 to 3.0% by weight, in particular 1.5 to 2.5% by weight, and also cells from the taxon Labyrinthulomycetes, in particular those of the family of Thraustochytriaceae, especially those of the genera Thraustochytrium, Schizochytrium, Aurantiochytrium or Oblongichytrium, in particular of the species Aurantiochytrium limacinum, especially the strain Aurantiochytrium limacinum SR21, where at least 80% by weight, in particular at least 90% by weight, preferably at least 95% by weight, of the particles present have a particle size of from 100 to 2000 micrometres, preferably 100 to 1500 micrometres, especially 100 to 1000 micrometres, where the biomass furthermore comprises hydrophilic or hydrophobic silicate in an amount of from 0.05 to 6% by weight, in particular 0.1 to 5% by weight, preferably 0.2 to 4.5% by weight, in particular 0.4 to 4.0% by weight, particularly preferably 0.6 to 3.5% by weight, in particular 0.8 to 3.2% by weight, especially 1.0 to 3.0% by weight, in particular 1.5 to 2.5% by weight.

Preference is given here in particular to a particulate biomass which comprises a modified cellulose, in particular carboxymethylcellulose, in an amount of from 0.05 to 6% by weight, in particular 0.1 to 5% by weight, preferably 0.2 to 4.5% by weight, in particular 0.4 to 4.0% by weight, particularly preferably 0.6 to 3.5% by weight, in particular 0.8 to 3.2% by weight, especially 1.0 to 3.0% by weight, in particular 1.5 to 2.5% by weight, and also cells of the genera Thraustochytrium, Schizochytrium, Aurantiochytrium or Oblongichytrium, in particular the species Aurantiochytrium limacinum, especially the strain Aurantiochytrium limacinum SR21, where at least 80% by weight, in particular at least 90% by weight, preferably at least 95% by weight, of the particles present have a particle size of from 100 to 2000 micrometres, preferably 100 to 1500 micrometres, especially 100 to 1000 micrometres, and which furthermore comprises a hydrophilic or hydrophobic silicate in an amount of from 0.05 to 6% by weight, in particular 0.1 to 5% by weight, preferably 0.2 to 4.5% by weight, in particular 0.4 to 4.0% by weight, particularly preferably 0.6 to 3.5% by weight, in particular 0.8 to 3.2% by weight, especially 1.0 to 3.0% by weight, in particular 1.5 to 2.5% by weight.

Preference is also given in particular to a particulate biomass which comprises a maltodextrin, in particular a maltodextrin with a DE value of 3 to 10, in an amount of from 0.05 to 6% by weight, in particular 0.1 to 5% by weight, preferably 0.2 to 4.5% by weight, in particular 0.4 to 4.0% by weight, particularly preferably 0.6 to 3.5% by weight, in particular 0.8 to 3.2% by weight, especially 1.0 to 3.0% by weight, in particular 1.5 to 2.5% by weight, and also cells of the genera Thraustochytrium, Schizochytrium, Aurantiochytrium or Oblongichytrium, in particular the species Aurantiochytrium limacinum, especially the strain Aurantiochytrium limacinum SR21, where at least 80% by weight, in particular at least 90% by weight, preferably at least 95% by weight, of the particles present have a particle size of from 100 to 2000 micrometres, preferably 100 to 1500 micrometres, especially 100 to 1000 micrometres, and which furthermore comprises a hydrophilic or hydrophobic silicate in an amount of from 0.05 to 6% by weight, in particular 0.1 to 5% by weight, preferably 0.2 to 4.5% by weight, in particular 0.4 to 4.0% by weight, particularly preferably 0.6 to 3.5% by weight, in particular 0.8 to 3.2% by weight, especially 1.0 to 3.0% by weight, in particular 1.5 to 2.5% by weight.

The particulate biomass according to the invention can be used in various ways. After drying of the biomass in accordance with the invention, the dried biomass is preferably stored or packed. Then, the biomass can for example be used on site in order to produce a foodstuff or feedstuff.

A feedstuff or foodstuff comprising a particulate biomass according to the invention is therefore a further subject matter of the present invention.

A further subject matter of the present invention is therefore likewise the use of a particulate biomass according to the invention for producing a foodstuff or feedstuff.

A further subject matter of the present invention is therefore likewise a method for producing a feedstuff or foodstuff, in which a particulate biomass according to the invention is used, and is preferably mixed with further feedstuff or foodstuff ingredients.

In order to increase the bioavailability of the PUFAs in the feedstuff or foodstuff to be produced, the particulate biomass can—directly before producing the feedstuff or foodstuff, be optionally subjected to a cell disruption method as described in the applications WO2014/122087 or WO2014/122092.

Alternatively, the biomass can, however, also be processed directly without prior cell disruption together with other feedstuff or foodstuff components to give a feedstuff or foodstuff.

A particulate biomass according to the invention is present here preferably in an amount of from 1 to 20% by weight, in particular 3 to 15% by weight, in a foodstuff or feedstuff according to the invention.

In an embodiment preferred according to the invention, the particulate biomass according to the invention is used for producing producing a foodstuff or feedstuff, in which the biomass is preferably mixed with other foodstuff or feedstuff ingredients and is then processed to give the foodstuff or feedstuff.

The mixture of biomass and other foodstuff or feedstuff ingredients is processed in a preferred embodiment by an extrusion process, in order to obtain portions of foodstuff or feedstuff ready for sale. Alternatively, a pelleting method may also be used, for example.

A screw or twin-screw extruder is preferably employed in the extrusion process. The extrusion process is preferably carried out at a temperature of 80-220° C., particularly 100-190° C., a pressure of 10-40 Bar, and a shaft rotational speed of 100-1000 rpm, particularly 300-700 rpm. The residence time of the mixture introduced is preferably 5-30 seconds, in particular 10-20 seconds.

In a mode of the extrusion process which is preferred in accordance with the invention, the process comprises a compacting step and a compression step.

It is preferred to intimately mix the components with each other before carrying out the extrusion process. This is preferably carried out in a drum equipped with vanes. In this mixing step, a preferred embodiment includes an injection of steam, in particular so as to bring about the swelling of the starch which is preferably present.

Before being mixed with the disrupted cells, the further foodstuff or feedstuff ingredients are preferably comminuted—if required—so as to ensure that a homogeneous mixture is obtained in the mixing step. The comminuting of the further foodstuff or feedstuff ingredients may be carried out, for example, using a hammer mill.

A method which is preferred in accordance with the invention for producing a foodstuff or feedstuff therefore comprises the following steps:

a) provision of a particular biomass which comprises a material of value, preferably a lipid, particularly preferably omega-3-fatty acids, preferably by spray granulation of a fermentation broth;

b) granulation of the resulting particulate biomass with the addition of an agglomeration auxiliary, where the granulation auxiliary used is preferably modified cellulose, in particular carboxymethylcellulose, or a maltodextrin, in particular a maltodextrin with a DE value of 3 to 10, such that the agglomeration auxiliary is present in the granulation product in an amount of from 0.1 to 6% by weight, preferably 0.2 to 5% by weight, particularly preferably 0.5 to 4% by weight;

c) mixing of the particulate biomass from (b), optionally after carrying out a cell disruption method beforehand, with further foodstuff or feedstuff ingredients;

d) producing the final product by a compacting or extrusion process.

A very particularly preferred method in accordance with the invention for producing a foodstuff or feedstuff comprises in this case the following steps:

a) provision of a particulate biomass which comprises slime nets, in particular those from the family of Thraustochytriaceae, especially those of the genera Thraustochytrium, Schizochytrium, Aurantiochytrium or Oblongichytrium;

b) granulation of the resulting particulate biomass with the addition of an agglomeration auxiliary, where the granulation auxiliary present is preferably modified cellulose, in particular carboxymethylcellulose, or a maltodextrin, in particular a maltodextrin with a DE value of 3 to 10, such that the agglomeration auxiliary is present in the granulation product in an amount of from 0.1 to 6% by weight, preferably 0.2 to 5% by weight;

c) mixing the particulate biomass from (b), optionally after carrying out a cell disruption method beforehand, with other foodstuff or feedstuff ingredients;

d) producing the final product by a compacting or extrusion process.

Methods preferred according to the invention for producing a foodstuff or feedstuff are preferably characterized in that the energy input to the biomass is no higher than 50 kWh per tonne of suspension in any method step. The energy input to the biomass is preferably at most 40 or 35 kWh, particularly at most 30 or 25 kWh, particularly preferably 20 or 15 kWh, in each case per tonne of suspension. This additionally ensures that the material of value present is adversely affected as little as possible.

The disrupted cells preferably account for 0.5-20% by weight, particularly 1-10% by weight, preferably 2-8% by weight of the foodstuff or feedstuff or of the composition used for producing the foodstuff or feedstuff.

The foodstuff or feedstuff is preferably a product for use in aquaculture or a foodstuff or feedstuff for use in poultry production, pig production or cattle production. The feedstuff may also take the form of a feedstuff which is employed for growing small organisms which may be employed as feedstuff in aquaculture. The small organisms may take the form of, for example, nematodes, crustaceans or rotifers. The feedstuff is preferably present in the form of flakes, spheres or tablets. A feedstuff obtainable by extrusion has a moisture content of preferably less than 5% by weight, especially preferably 0.2 to 4% by weight.

The other foodstuff or feedstuff ingredients are preferably selected from protein-containing, carbohydrate-containing, nucleic-acid-containing and lipid-soluble components and, if appropriate, further fat-containing components and furthermore from among other additives such as minerals, vitamins, pigments and amino acids. In addition, structurants may also be present, besides nutrients, for example so as to improve the texture or the appearance of the feedstuff. Furthermore, it is also possible to use, for example, binders so as to influence the consistency of the feedstuff. A component which is preferably employed and which constitutes both a nutrient and a structurant is starch.

The following examples may be employed as a protein-containing component which additionally contains fats: fish meal, krill meal, mussel meal, squid meal or shrimp shells. As an alternative, fish oil may also be used as a fat-containing component. A vegetable oil may also be employed as a fat-containing component, in particular oil from soybeans, rapeseed, sunflower kernels and flaxseeds. An example of a carbohydrate-containing component which may be employed is wheat meal, sunflower meal, soya meal or cereal gluten.

The total oil content in the feedstuff—including the oil from the oil-containing cells—amounts preferably to 15-50% by weight.

The feedstuff for use in aquaculture is preferably used for breeding finfish and crustaceans which are preferably intended for human nutrition. These include, in particular, carp, tilapia, catfish, tuna, salmon, trout, barramundi, bream, perch, cod, shrimps, lobster, crabs, prawns and crayfish. It is especially preferably a feedstuff for salmon farming. Preferred types of salmon in this context are the Atlantic salmon, red salmon, masu salmon, king salmon, keta salmon, coho salmon, Danube salmon, Pacific salmon and pink salmon.

Alternatively, it may also be a feedstuff intended for farming fish which are subsequently processed to give fish meal or fish oil. These fish are preferably herring, pollock, menhaden, anchovies, caplin or cod. The fish meal or fish oil thus obtained, in turn, can be used in aquaculture for farming edible fish or crustaceans.

Aquaculture may take place in ponds, tanks, basins or else in segregated areas in the sea or in lakes, in particular in this case in cages or net pens. Aquaculture may be used for farming the finished edible fish, but also may be used for farming fry which are subsequently released so as to restock the wild fish stocks.

In salmon farming, the fish are preferably first grown into smolts in freshwater tanks or artificial watercourses and then grown on in cages or net pens which float in the sea and which are preferably anchored in bays or fjords.

Accordingly, a further subject matter of the present invention is also a method for farming animals, in particular finfish or crustaceans, preferably salmon, in which a feedstuff according to the invention is used. A further subject matter of the present invention is additionally an animal, in particular a finfish or shellfish, which is obtainable by such a method according to the invention.

### WORKING EXAMPLES

### Example 1

### Production of Spray-Dried Biomass

To produce DHA, the strain Aurantiochytrium limacinum SR21 was used. This is deposited at the NIBH under FERM BP-5034 and also at the IFO under IFO 32693. The strain A. limacinum SR21 was originally isolated from seawater and called Schizochytrium limacinum SR21 (Nakahara et al. 1996, JAOCS, 73(10); Honda Mycol. Res. 1998). On account of the new classification, it was assigned to the new genus Aurantiochytrium and renamed accordingly.

The fermentation of the strain was carried out in a medium which comprise 50% synthetic seawater (Sigma Aldrich) and furthermore comprise the following components: 60 g/l glucose, 0.7 g/l corn steep liquor (Sigma Aldrich), 2 g/l (NH₄)₂SO₄ and 3 g/l KH₂PO₄.

The fermentation was carried out at 28° C., a pH of 4.0, an aeration rate of 0.5 vvm and a stirring of 200 rpm for 60 hours. After ending the fermentation, an antioxidant was added to the fermentation broth and the fermentation broth was then heated at 60° C. for at least 20 minutes.

Then, a two-stage drying of the biomass was carried out: Firstly, the fermentation broth was concentrated to a dry mass of about 20% by weight by evaporation. Then, spray drying of concentrated fermentation broth was carried out using a Production Minor™ Spray Dryer (GEA NIRO) at an inlet temperature of the drying air of 340° C. Spray drying produced a powder with a dry mass of more than 95% by weight.

### Example 2

### Granulation of Spray-Dried Biomass Using Carboxymethylcellulose as Agglomeration Auxiliary

For the agglomeration of the spray-dried particles from Example 1, the granulator ProCell-LabSystem (Glatt, Germany) was used to carry out a fluidized-bed granulation. Here, the insert GF3 was used. The fine, spray-dried biomass powder was fed to the fluidized bed and a carboxymethylcellulose solution (4% by weight blanose, dissolved in water) was sprayed in and thus finely distributed in the fluidized bed such that a final concentration of carboxymethylcellulose of 3% by weight was established. The solvent was evaporated by heating the incoming air to 60° C. The incoming air rate was adjusted such that a fluidization of the agglomerated particles is possible. This was achieved by regulating the amount of incoming air to about 80 m³/h. As soon as the agglomerates have reached the desired particle size, they were no longer fluidized on account of their weight and were able to be removed at the lower end of the fluidized bed. The residence time of the particles in the fluidized bed was thus established via the rate of particle growth since the particles had to grow from fine, spray-dried particles to the relatively large agglomerate in order to leave the fluidized bed.

The pregiven conditions corresponded to the standard conditions of a thermal granulation.

The product thus obtained exhibited, compared to the starting biomass, significantly improved product properties, in particular a considerably improved free-flowability.

### Example 3

### Granulation of Spray-Dried Biomass Using Maltodextrin DE 3.5 as Agglomeration Auxiliary

For the agglomeration of the spray-dried particles from Example 1, the granulator ProCell-LabSystem (Glatt, Germany) was used to carry out a fluidized-bed granulation. Here, the insert GF3 was used. The fine, spray-dried biomass powder was fed to the fluidized bed, and a maltodextrin DE 3.5 solution (25% by weight maltodextrin DE 3.5, dissolved in water) was sprayed in and thus finely distributed in the fluidized bed, such that a final concentration of maltodextrin DE 3.5 of 3% by weight was established. The solvent was evaporated by heating the incoming air to 60° C. The incoming air rate was adjusted such that a fluidization of the agglomerated particles is possible. This was achieved by regulating the amount of incoming air to about 80 m³/h. As soon as the agglomerates have reached the desired particle size, these were no longer fluidized on account of their weight and could be removed at the bottom end of the fluidized bed. The residence time of the particles in the fluidized bed was thus established by the rate of particle growth since the particles had to grow from the fine, spray dried particle to the relatively large agglomerate in order to leave the fluidized bed.

The pregiven conditions corresponded to the standard conditions of a thermal granulation.

The product obtained in this way exhibited, compared to the starting biomass, considerably improved product properties, in particular a considerably improved free-flowability.

### Example 4

### Granulation of Spray-Dried Biomass Using Maltodextrin 18.9 as Agglomeration Auxiliary

For the agglomeration of the spray-dried particles from Example 1, the granulator ProCell-LabSystem (Glatt, Germany) was used for carrying out a fluidized-bed granulation. Here, the insert GF3 was used. The fine, spray-dried biomass powder was fed to the fluidized bed, and a maltodextrin 18.9 solution (40% by weight maltodextrin 18.9, dissolved in water) was sprayed in and thus finely distributed in the fluidized bed, such that a final concentration of maltodextrin DE 18.9 of 3% by weight was established. The solvent was evaporated by heating the incoming air to 60° C. The incoming air rate was adjusted such that a fluidization of the agglomerated particles is possible. This was achieved by regulating the amount of incoming air to about 80 m³/h. As soon as the agglomerates have reached the desired particle size, these were no longer fluidized on account of their weight and could be removed at the bottom end of the fluidized bed. The residence time of the particles in the fluidized bed was thus established by the rate of particle growth since the particles had to grow from the fine, spray dried particle to the relatively large agglomerate in order to leave the fluidized bed.

The pregiven conditions corresponded to the standard conditions of a thermal granulation.

The product obtained in this way exhibited, compared to the starting biomass, considerably improved product properties, in particular a considerably improved free-flowability.

1-17. (canceled)

18. A method for producing a particulate biomass having an oxidation-sensitive material of value, said method comprising granulating a particulate starting biomass in the presence of an agglomeration auxiliary.

19. The method of claim 18, wherein carbohydrates, or modified carbohydrates, are used as the agglomeration auxiliary.

20. The method of claim 18, wherein polysaccharides, or modified polysaccharides, are used as the agglomeration auxiliary.

21. The method of claim 18, wherein the agglomeration auxiliary is selected from the group consisting of: guaran; gum Arabic; guar gum; locust bean gum; xanthan gum; agar; carrageenan; a maltodextrin; starch; cellulose or its derivatives; hemicellulose or its derivatives; alginic acid; maltodextrin; and mixtures thereof.

22. The method of claim 21, wherein said agglomeration auxiliary is selected from the group consisting of: modified cellulose; maltodextrin; and mixtures thereof.

23. The method of claim 18, wherein proteins are used as the agglomeration auxiliary.

24. The method of claim 18, wherein organic polymers are used as the agglomeration auxiliary.

25. The method of claim 18, wherein inorganic substances are used as the agglomeration auxiliary.

26. The method of claim 18, wherein the starting biomass is obtained by spray drying a fermentation broth containing the biomass.

27. The method of claim 18, wherein the biomass comprises cells from the taxon Labyrinthulomycetes.

28. The method of claim 18, wherein the oxidation-sensitive material of value is a PUFA.

29. The method of claim 18, wherein:
a) the agglomeration auxiliary is selected from the group consisting of: guaran; gum Arabic; guar gum; locust bean gum; xanthan gum; agar; carrageenan; a maltodextrin; cornstarch; tapioca starch; potato starch,; cellulose or its derivatives; hemicellulose or its derivatives; alginic acid; maltodextrin; casein, gelatine, collagen; wheat gluten; lignosulphonate; polymethylolcarbamides; polyacrylic acids; polyvinyl alcohols; bentonites; hexametaphosphate and mixtures thereof;
b) the particulate starting biomass has been obtained by spray drying a fermentation broth containing the biomass;
c) the biomass comprises cells from family Thraustochytriaceae;
d) the oxidation-sensitive material of value is an omega-3-fatty acid.

30. A particulate biomass having an oxidation-sensitive material of value, comprising an agglomeration auxiliary in an amount of from 0.05 to 6% by weight.

31. The particulate biomass of claim 30, wherein the agglomeration auxiliary is a carbohydrate or a modified carbohydrate.

32. The particulate biomass of claim 30, wherein said biomass comprises a hydrophilic or hydrophobic silicate in an amount of from 0.05 to 6% by weight.

33. The particulate biomass of claim 30, wherein at least 80% by weight of the particles have a particle diameter (grain size) of from 100 to 2000 μm.

34. The particulate biomass claim 30, wherein said biomass comprises cells from the taxon Labyrinthulomycetes.

35. The particulate biomass of claim 30, wherein the oxidation-sensitive material of value is a PUFA.

36. The particulate biomass of claim 30, comprising an agglomeration auxiliary in an amount of from 0.4 to 4% by weight, wherein:
a) the agglomeration auxiliary is selected from the group consisting of: carboxymethylcellulose; hydroxypropylmethylcellulose, maltodextrin; and a hydrophilic or hydrophobic silicate in an amount of from 0.1 to 5% by weight;
b) at least 90% by weight of the particles have a particle diameter (grain size) of from 100 to 1000 μm;
c) the biomass comprises cells selected from one or more genera selected from the group consisting of: Thraustochytrium; Schizochytrium; Aurantiochytrium; Oblongichytrium; and Ulkenia; and
d) the oxidation-sensitive material of value is an omega-3-fatty acid.

37. A feedstuff or foodstuff comprising the particulate biomass of claim 30.
